(19) United States
(12) Patent Application Publication
(10) Pub. No.: US 2019/0336318 A1
(43) Pub. Date: Nov. 7, 2019

(54) MENSTRUAL CUP

(71) Applicant: IMARI CO., LTD., Saga-shi, Saga (JP)

(72) Inventor: Masaya KUBO, Saga (JP)

(73) Assignee: Imari Co., Ltd., Saga-shi, Saga (JP)

(21) Appl. No.: 16/300,441

(22) PCT Filed: Sep. 20, 2017
(86) PCT No.: PCT/JP2017/033906

§ 371 Date: Nov. 9, 2018

(30) Foreign Application Priority Data
Jan. 27, 2017 (JP) 2017-013674

(51) Int. Cl. A61F 5/455

(52) U.S. Cl. CPC A61F 5/4553

(57) ABSTRACT

A high-quality menstrual cup that can be used repeatedly and is comfortable when used. The menstrual cup has a head portion, a body portion, and a handle portion that are formed of the same silicone resin. This silicone resin has sufficient elasticity to permit the above portions to be deformable upon application of an external holding force by a user's fingers as well as structural restoration when the applied external force is removed. The head portion and the body portion are integrally formed, and a receiving space having a bottom is formed inside the head portion and the body portion. The receiving space has an opening on the upper side that allows menstrual blood to flow inward from the the opening (6), so that the menstrual blood can be received in the receiving space (5).

### CROSS-REFERENCE TO RELATED APPLICATIONS

This application is based on and claims the benefit of priority to Japan Patent Application No. 2017-013674 filed on Jan. 27, 2017, with the Japan Patent Office, the disclosure of which is incorporated herein by reference.

### BACKGROUND OF THE INVENTION

### Field of the Invention

The present invention relates to a menstrual cup. More specifically, the present invention relates to a high-quality menstrual cup which can be repeatedly used and is excellent in feeling of use.

### Description of the Prior Art

Conventionally, menstrual articles such as a menstrual napkin have been used to cope with menstruation, which is a physiological bleeding of a woman.

The menstrual napkin has a structure in which an absorber, which is made of a resin or the like, for absorbing menstrual blood, is disposed inside the sheet-shaped main body. In addition, various measures have been made to prevent menstrual blood from easily leaking due to the user's movement or due to the amount of menstrual blood of to improve wearing feeling when using such a menstrual napkin.

For example, in the menstrual napkin disclosed in Patent Document 1, a coarse-dense structure composed of a high-density portion and a low-density portion is provided along the longitudinal direction of the absorber on the rear portion of the napkin, thereby improving liquid penetration and wearability of the rear portion of the napkin.

### CITATION LIST

### Patent Literature

Patent Document 1: Japanese Patent Publication No. 2011-120697

### SUMMARY OF THE INVENTION

### Technical Problem

However, in the menstrual napkins including the menstrual napkin disclosed in Patent Document 1, leakage may occur depending on the amount of menstrual blood or the user's movement. Skin diseases, which are caused by leakage of menstrual blood or the contact of leaked menstrual blood to the skin, give the user a great discomfort.

Further, since it is difficult to cope with the occurrence of leakage during long-time outside activity or sleeping, the user is more likely to feel uncomfortable.

Also, depending on the temperature or the weather, for example, when it is highly humid or hot, during the use of a menstrual napkin, odor may be produced and the user may experience sweatiness, itchiness, and the like. This inconvenience is difficult to avoid even if the menstrual blood is able to be absorbed by the sanitary napkin.

In addition, there is also a problem in that since the menstrual napkins are disposable articles, the user pays a lot of money for use thereof.

The present disclosure has been made in view of the above points, and relates to a high-quality menstrual cup, which can be repeatedly used and has excellent feeling of use.

### Solution to Problem

In order to achieve the aspects described above, the menstrual cup having a bell-shaped main body having a bottomed bore portion of the present invention includes: a body portion including an opening formed in one end opposite to a bottom portion of the main body; a head portion provided to be continued to the one end of the body portion and having a shape, of which an outer diameter increases in a direction opposite to the body portion; an inner wing portion provided over an entire inner wall of a distal end portion of the head portion and protruding towards the bottom portion in the inner direction; and a handle portion provided at the other end of the body portion and protruding in a direction opposite to the body portion by a predetermined amount.

Here, since the body portion has a bell-shaped main body having a bottomed-bore portion formed therein, and an opening is formed at one end opposite the bottom portion of the main body, menstrual blood can be introduced from the opening and a predetermined amount of menstrual blood can be collected in the bore portion. In addition, since the menstrual blood is collected inside the body portion so as not to come into contact with the skin and the vagina of the user, sweating, skin diseases, and the like are not easily caused during use. Further, after use of the menstrual cup, the menstrual blood collected from the opening can be disposed.

In addition, since the body portion has a bell-shaped main body, when the menstrual cup is inserted into the vagina of the user, the outer peripheral surface of the body portion comes into smooth contact with the inner wall of the vagina of the user. Thus, the menstrual cup can be held in the vagina while minimizing the stimulation to the inner wall of the vagina. In addition, since the menstrual cup can be held in the vagina, the menstrual blood collected inside the body portion does not easily come into contact with air, and thus, the smell generated when the menstrual blood is in contact with oxygen can be reduced.

In addition, since the head portion is provided to be continued to the one end of the body portion and has a shape in which the outer diameter increases in the direction opposite to the body portion, not only the outer peripheral surface of the body portion, but also the outer peripheral surface of the head portion having a larger outer diameter is brought into contact with the inner wall of the vagina, thereby making it easy to hold the menstrual cup in the vagina.

In addition, by the head portion provided to be continued to the one end of the body portion and having a shape of which the outer diameter is increased in the direction opposite the body portion, and the inner wing portion provided on the entire inner wall of the distal end portion of the head portion and protruding inwards and towards the bottom portion, the menstrual blood generated in the vagina flows easily towards the inside of the body portion along the head portion and the inner wing portion, so that the menstrual blood can be easily collected inside the body portion. Further, since the movement of the menstrual blood collected inside the body portion, which tends to flow out to the outside of the body portion, i.e. from the opening to the outside, is blocked by the inner wing portion, it is possible to suppress the menstrual blood from leaking out of the menstrual cup once the menstrual blood is collected inside the body portion.

Further, since the handle portion is provided at the other end of the body portion and protrudes in a direction opposite the body portion by a predetermined amount, the insertion position of the menstrual cup can be easily confirmed by touching the handle portion with a finger when the menstrual cup is held in the vagina of the user.

Further, when a through hole penetrating from the outer peripheral surface to the inner surface is formed in at least a portion of the body portion or the head portion, it is possible to draw out the air in the region in front of the menstrual cup in the vagina in the insertion direction of the menstrual cup when inserting the menstrual cup into the vagina. This makes it easy to insert the menstrual cup into the vagina. In addition, when the amount of menstrual blood collected in the body portion is increased such that the menstrual blood is collected to the vicinity of the position where the through hole is formed, a bubbling sound may be produced in the portion in which the through hole is formed or the user may be caused to feel vibration at that time. Then, the user is capable of determining that a predetermined amount of menstrual blood is collected, thereby estimating the timing at which the menstrual cup is taken out from the vagina. In addition, if the menstrual blood is collected beyond the position of the through hole, the menstrual blood leaks through the through hole. Thus, the user is capable of determining the timing to take out the menstrual cup from the vagina based on this leakage.

Further, when a plurality of convex portions are formed along the longitudinal direction on the outer peripheral surface of the handle portion, it becomes easier for the user to recognize the handle portion by touching the handle portion with a finger. Thus, it becomes easier to confirm the insertion position of the menstrual cup during the insertion thereof.

In addition, when the convex portions are formed in a region of the handle portion at the side of the body portion and corresponding to two thirds of the length of the handle portion in the longitudinal direction, the convex portions are located in the region, closer to the body portion, of the handle portion. Thus, when the menstrual cup is held in the vagina, the inner wall of the vagina and the convex portions on the handle portion can be easily brought into contact with each other. Therefore, it is possible to reduce discomfort during the use of the menstrual cup.

When the ratio of the length of the handle portion in the longitudinal direction to the length of the body portion and the head portion in the longitudinal direction is in the range of 1:3.0 to 4.5, the handle portion has an appropriate length with respect to the entire size of the menstrual cup. Thus, it is possible to reduce discomfort during use of the menstrual cup while ensuring the function of facilitating the confirmation of the insertion position of the menstrual cup.

Here, when the ratio of the length of the handle portion in the longitudinal direction and the length of the body portion and the head portion in the longitudinal direction is less than 3.0, the handle portion is shortened, and thus, it becomes difficult to confirm the insertion position of the menstrual cup. In addition, when the ratio of the length of the handle portion in the longitudinal direction to the length of the body portion and the head portion in the longitudinal direction exceeds 4.5, the handle portion may become unnecessarily long, and the feeling of use may deteriorate.

Further, in the case where the rib portions are provided on the outer peripheral surface of the other end of the body portion and protrude in a direction opposite the body portion, the user is able to confirm the insertion position of the menstrual cup by touching the rib portions with a finger. That is, it is also possible to confirm the insertion position of the menstrual cup not based on the handle portion but also based on the rib portions.

Further, when a cutout portion is formed in at least a portion of the distal end portion of the inner wing portion, the menstrual blood collected inside the body portion is able to flow out from the cutout portion, and the menstrual blood can be easily discarded after using the menstrual cup.

### Effects of the Invention

A menstrual cup according to the present disclosure is repeatedly usable, and is of high quality with excellent feeling of use.

### BRIEF DESCRIPTION OF THE DRAWINGS

The above and other aspects, features and advantages of the present disclosure will be more apparent from the following detailed description taken in conjunction with the accompanying drawings, in which:

FIG. 1A is a front view illustrating a menstrual cup to which the present disclosure is applied, and FIG. 1B is a top plan view of the menstrual cup of FIG. 1A;

FIG. 2A is a schematic perspective view of the menstrual cup of FIG. 1A, FIG. 2B is a cross-sectional view of the menstrual cup of FIG. 1A, and FIG. 2C is a schematic perspective view illustrating the menstrual cup of FIG. 1A partially in cross section;

FIG. 3A is a front view of the menstrual cup of FIG. 1A, and FIG. 3B is an enlarged view of a region indicated by reference numeral X₁ in FIG. 3A;

FIG. 4A is a rear view of the menstrual cup illustrated in FIG. 3A, and FIG. 4B is an enlarged view of a region indicated by reference numeral X₂ in FIG. 4A; and

FIGS. 5A and 5B are a schematic view and a schematic cross-sectional view for explaining a method of using a menstrual cup to which the present disclosure is applied.

### DETAILED DESCRIPTION OF THE EXEMPLARY EMBODIMENTS

Hereinafter, embodiments of the present disclosure will be described with reference to the drawings, in which the embodiments are provided for helping the understanding of the present disclosure.

In addition, a structure described below is merely an example of a menstrual cup to which the present disclosure is applied, and the contents of the present disclosure are not limited thereto and can be appropriately changed.

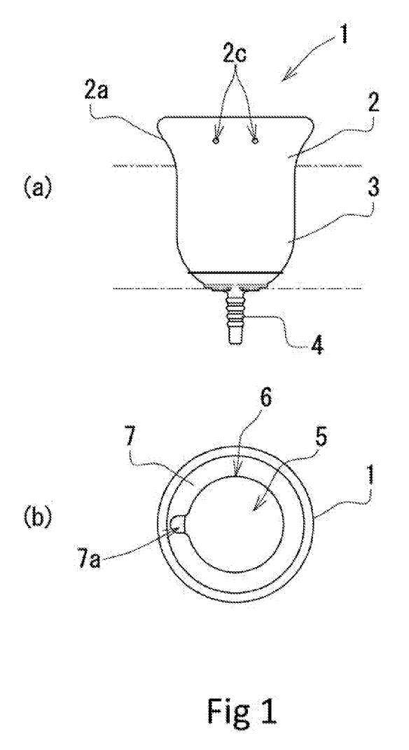

As illustrated in FIG. 1, a menstrual cup 1, which is an example of a menstrual cup according to the present disclosure, is provided with a head portion 2, a body portion 3, and a handle portion 4.

In the embodiments of the present disclosure described below, the position of the handle portion 4 with respect to the head portion 2 is indicated by a term “lower” or “downward”, and the position of the head portion 2 relative to the handle portion 4 is indicated by a term “upper” or “upward”. In addition, the region of a receiving space 5 formed inside the head portion 2 and the body portion 3 to be described below will be indicated by a term “inner” or “inside”, and the region of the outer peripheral surfaces of the head portion 2 and the body portion 3 will be indicated by a term “outer” or “outside”. The direction from the position of the head portion 2 toward the handle portion 4 or the direction from the position of the handle 4 toward the head portion 2 will be referred to as a “vertical direction” or a “longitudinal direction”.

The head portion 2 and the body portion 3 form a portion having a shape for collecting menstrual blood in the menstrual flow cup 1. The handle portion 4 is a portion for confirming the position of the cup when the menstrual cup 1 is inserted into the vagina. The head portion 2, the body portion 3, and the handle portion 4 are integrally formed using the same silicone resin. The outer peripheral surfaces of the head portion 2 and the body portion 3 are formed smoothly. In addition, the silicone resin has appropriate elasticity and is deformable by a holding force applied by fingers, and has a structure to be restored when an external force applied thereto is removed. In addition, the silicone resin has heat resistance that can withstand disinfection by boiling in hot water or the like.

As illustrated in FIGS. 1A and 1B, the head portion 2 and the body portion 3 are integrally formed, and a receiving space 5 having a bottom is formed inside the head portion 2 and the body portion 3. In addition, the body portion 3 has a generally bell-shaped outer shape. The receiving space 5 has an opening 6 on the upper side and the menstrual blood flows inwards from the portion of the opening 6, so that the menstrual blood can be collected in the receiving space 5. The volume of the receiving space 5 has a size capable of collecting about 25 ml of liquid.

The head portion 2 has a smooth outer peripheral surface provided with a taper 2a, of which the outer diameter increases toward the upper end (see FIG. 1A). The outer diameter of the head portion 2 is determined so as to be larger than the outer diameter of the body portion 3. Further, the outer peripheral surface of the head portion 2 is formed in a slightly rounded and smooth shape by being chamfered. Further, the outer peripheral surface of the body portion 3 is formed in a smooth shape by being chamfered.

As described above, the head portion 2, the body portion 3, and the handle portion 4 are integrally formed, and there is no clear boundary between the portions. However, for convenience of explanation, the upper portion above the portion, of which the outer diameter increases (the uppermost region among the regions divided by one-dot chain lines indicated in FIG. 1A) is referred to as the head portion 2, the portion having a bell-shaped external appearance (the central region among the regions divided by one-dot chain lines indicated in FIG. 1A) is referred to as the body portion 3, and the lower portion, of which the outer diameter decreases (the lowermost region among the portions divided by one-dot chain lines indicated in FIG. 1A) is referred to as the handle portion 4.

The total length of the menstrual cup 1 in its longitudinal direction is about 57.8 mm. In addition, the length of the combined portion of the head portion 2 and the body portion 3 in the longitudinal direction is about 45 mm and the length of the handle portion 4 in the longitudinal direction is about 13 mm. In addition, the diameter of the head portion 2 at the greatest outer diameter is 40 mm.

Two air holes 2c (corresponding to through holes in the claims of the present application) are formed on the upper side of the body portion 3 (see FIG. 1A). The position where the air holes 2c are provided is a substantially central position of the head portion 2 in the vertical direction. The air holes 2c are through holes each having a diameter of 1.5 mm and penetrating from the outer peripheral surface to the inner peripheral surface of the body portion 3 and serve as a portion for venting the air inside the vagina when the menstrual cup 1 is inserted thereinto. Further, the air holes 2c serve as a portion for discharging the menstrual blood when the menstrual blood collected in the receiving space 5 is collected beyond the position of the air holes 2c.

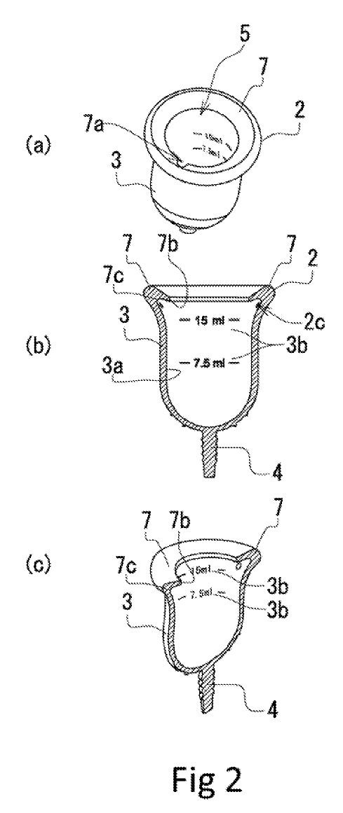

In FIG. 1A, two air holes 2c are illustrated, but in the menstrual cup, a total of four air holes 2c may be formed in the head portion 2. That is, two air holes 2c also are formed in the head portion 2 at positions opposite the air holes 2c illustrated in FIG. 1A (see FIGS. 2B, 3A, and 4A).

Here, the head portion 2, the body portion 3, and the handle portion 4 are not necessarily formed of the same silicone resin, but may be partially formed of different materials. However, the head portion 2, the body portion 3, and the handle portion 4 are preferably formed of the same silicone resin since they can be integrally manufactured using a mold or the like by adopting the same material.

In addition, the head portion 2, the body portion 3, and the handle portion 4 are not necessarily formed of the silicone resin. However, it is preferable that the head portion 2, the trunk portion 3, and the handle portion 4 are formed of a silicone resin for the following reasons: the silicon resin is excellent in durability, allows disinfection by boiling, and is capable of being repeatedly used; the silicon resin has appropriate elasticity so as to facilitate insertion of the menstrual cup 1 into the vagina; and the silicon resin allows the shape of the menstrual cup 1 to be changed according to the movement of the user's body when the menstrual cup 1 is held in the vagina, thereby making the wearablity of the menstrual cup 1 excellent.

In addition, the volume of the receiving space 5 in the menstrual cup 1 does not need to be limited to a size capable of collecting liquid of about 25 ml. The volume of the receiving space 5 or the sizes of the head portion 2 and the body portion 3 forming the receiving space 5 may be appropriately set and changed as necessary. However, for example, Japanese women are assumed to be the target users, it is preferable that the volume of the receiving space 5 is set to a size capable of collecting about 25 ml of liquid in consideration of the ease of insertion into the vagina of a user and the feeling of use when the menstrual cup 1 is held in the vagina, and it is preferable that the head portion 2 and the body portion 3 have a size for implementing this.

In addition, the number of the air holes 2c provided in the menstrual cup 1, the formation positions of the air holes 2c, and the sizes of the air holes are not particularly limited so long as the air holes 2c are provided such that when inserting the menstrual cup 1 into the vagina of the user, the air holes 2c allow the air in the inner region to be removed from the menstrual cup 1 in the vagina, thereby making the menstrual cup 1 insertable into the vagina. The number, the formation positions, and the size of the hole of the air holes 2c may be appropriately set and changed in consideration of molding stability during the manufacture of the menstrual cup 1 and the durability of the menstrual cup 1. However, it is preferable that a total of four air holes 2c (two holes on each side) are formed in the center in the vertical direction of the head portion 2 for the following regions: the air in the vagina is easily removed from the air holes 2c, thereby facilitating insertion of the menstrual cup 1 into the vagina; and it becomes easy to determine a predetermined amount of collected menstrual blood, and an excessive amount of menstrual blood is efficiently discharged.

In addition, the size of the menstrual cup 1 does not have to be limited to the above-mentioned sizes. For example, the menstrual cup 1 may have different sizes depending on the feeling of use, the wearability, the amount of menstrual blood, etc. when the user holds the menstrual cup in the vagina.

In addition, the combined length of the head portion 2 and the body portion 3 in the longitudinal direction does not have to be limited to 45 mm, and the length of the handle portion 4 in the longitudinal direction does not have to be limited to 13 mm. However, when the ratio of the height of the handle portion to the height of the body portion and the head portion is within the range of 1:3.0 to 4.5, the handle portion is appropriately sized so as not to be too short or too long, so that the feeling of use can be improved while ensuring the function of facilitating the confirmation of the position of the cup.

As illustrated in FIG. 1A and FIGS. 2A to 2C, on the inner peripheral surface side of the head portion 2, an inner wing portion 7 is formed along the entire circumference of the inner peripheral surface of the head portion 2. The inner wing portion 7 is a structure for inhibiting the menstrual blood collected in the receiving space 5 from flowing out to the outside of the menstrual cup 1. In addition, the inner wing portion 7 also serves as a guide portion for facilitating the flow of menstrual blood in the vagina into the receiving space 5.

A cutout portion 7a is formed in a portion of the inner peripheral surface of the inner wing portion 7 (see FIGS. 1B and 2A). As illustrated in FIG. 1B, the cutout portion 7a is formed in a substantially U-shape in plan view.

As illustrated in FIG. 2B, the inner wing portion 7 has a distal end portion 7b protruding inwards and downwards, that is, toward the receiving space 5 in an obliquely downward direction. In addition, the distal end portion 7b of the inner wing portion 7 is located further inside than the position of the inner peripheral surface 3a of the body portion 3. The upper end surface of the inner wing portion 7 is formed in a tapered shape directed obliquely downwards.

Since the inner wing portion 7 is projected obliquely downward toward the accommodation space 5, even when the menstrual cup 1 is inclined obliquely from the horizontal direction, the menstrual blood collected in the receiving space 5 is easily stopped at the portion of the inner wing portion 7. That is, the inner wing portion 7 serves to block the menstrual blood collected inside the menstrual cup 1. Further, the inner wing portion has a shape that is capable of easily guiding the menstrual blood from the outside of the menstrual cup 1 to the inner receiving space 5.

The inner wing portion 7 has an angle of about 40 degrees with the upper end surface of the menstrual cup 1 when viewed in side view, the length of the inner wing portion 7 in the vertical direction from the upper end surface of the cup 1 is set to about 4.8 mm, and the length of the inclined surface of the inner wing portion 7 is set to about 7.0 mm.

In addition, the distal end portion 7b of the inner wing portion 7 is formed to have a thickness of about 1.0 mm and the strength of the inner wing portion 7 is improved. In addition, the distal end portion 7b is chamfered to have a rounded shape. A return portion 7c located at a position where the lower end surface of the inner wing portion 7 and the inner wall surface 3a of the head portion 2 intersect is formed as a curved surface, and the inner wing portion 7 is hindered from tearing the return portion 7c as a starting point (See FIG. 2B).

Here, the shape and size of the inner wing portion 7, the angle of the inclined surface, and the like are not limited to the above-mentioned ones and may be appropriately set.

As illustrated in FIGS. 2B and 2C, on the inner peripheral surface of the body portion 3, two types of reference indication portions 3b including lines having a predetermined length and character information are provided at predetermined height positions. The reference indication portions 3b indicate the amounts of menstrual blood when the menstrual blood is collected to the height positions of the lines.

The user is capable of taking out the menstrual cup 1 from the vagina, and easily confirming the amount of collected menstrual blood through the reference indication portions 3b.

Here, the reference indication portions 3b do not have to be provided on the inner peripheral surface of the body portion 3. However, it is preferable that the reference indication portions 3b is provided on the inner peripheral surface of the body portion 3 in view of the fact that the user can easily confirm the amount of menstrual blood collected in the menstrual cup 1. In addition, the reference indication portions 3b need not be limited to the two types, and the number of the indication portions, the indication amounts, and the formation positions may be appropriately changed as necessary.

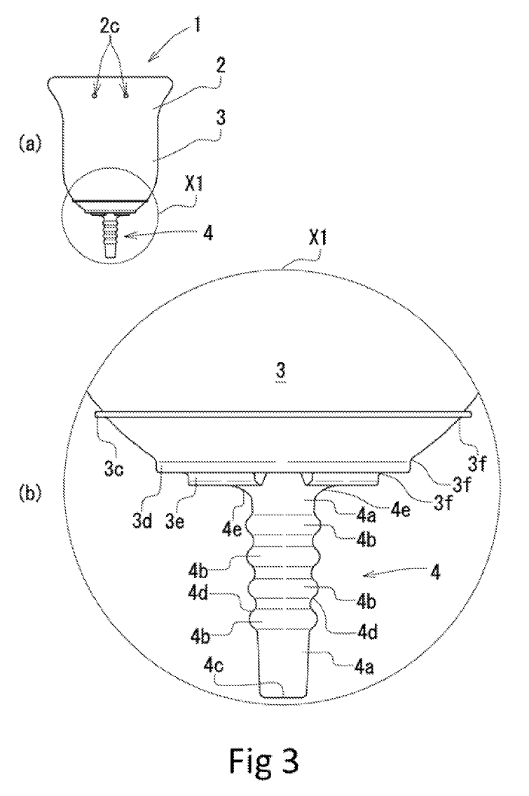

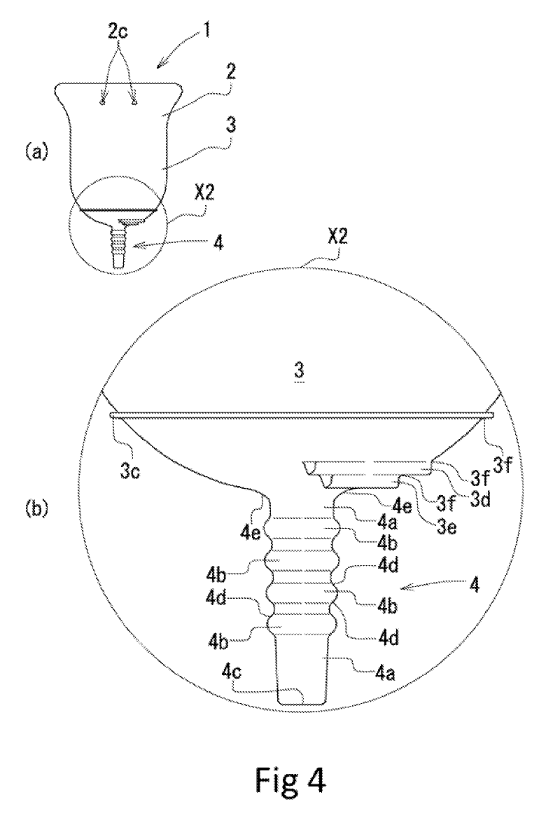

The handle portion 4 and the structures therearound will be described with reference to FIGS. 3A and 3B and FIGS. 4A and 4B. FIGS. 3A and 3B illustrate the structure of the menstrual cup 1 when viewed from the front side of the menstrual cup 1, and FIGS. 4A and 4B illustrate the structure of the menstrual cup 1 when viewed from the rear side of the menstrual cup 1.

The handle portion 4 is a member which is provided substantially at the center of the bottom surface of the body portion 3 and protrudes downwards by a predetermined length (see FIGS. 3A and 3B and FIGS. 4A and 4B). The handle portion 4 is spaced apart from the receiving space 5 of the body portion 3, and is a portion for confirming the position of the menstrual cup 1 when the menstrual cup 1 is held in the vagina as described above (see FIGS. 2B and 2C).

The handle portion 4 includes a substantially cylindrical columnar portion 4a extending in the longitudinal direction and four large-diameter portions 4b provided at regular intervals in the longitudinal direction of the columnar portion 4a (see FIGS. 3B and 4B). The large-diameter portions 4b correspond to convex portions in the claims of the present application. The columnar portion 4a of the handle portion 4 is a solid member (see FIGS. 2B and 2C).

The outer diameter of the large-diameter portions 4b is larger than the outer diameter of the columnar portion 4a (see FIGS. 3B and 4B). The user is able to confirm the position of the menstrual cup 1 retained in the vagina by touching the columnar portion 4a or the plurality of large-diameter portions 4b provided on the columnar portion 4a with a finger.

The large-diameter portions 4b are provided in a region nearer to the body portion 3 in the longitudinal direction of the columnar portion 4a, and in the range of about two thirds of the length in the longitudinal direction of the columnar portion 4a.

In the handle portion 4, the outer peripheral surfaces of the large-diameter portions 4b and the distal end portion 4c of the columnar portion 4a are chamfered to form smooth rounded shapes. This makes it possible to reduce the discomfort felt by the user even when the outer peripheral surfaces of the large-diameter portions 4b or the distal end portion 4c of the columnar portion 4a touches the inner wall of the vagina or the like.

In addition, transition portions 4d between the columnar portion 4a and the large-diameter portions 4b and a transition portion 4e between the columnar portion 4a and the bottom surface of the body portion 3 are formed as curved surfaces. As a result, the occurrence of tearing at the transition portion 4e can be suppressed, and the durability can be improved.

Here, the handle portion 4 does not have to include the columnar portion 4a and the large-diameter portions 4b. However, it is preferable that the handle portion 4 includes the columnar portion 4a and the large diameter portions in view of the fact that it is easy for the user to detect the handle portion 4 by touching the handle portion 4 with a finger and it is easy to confirm the position of the menstrual cup 1 in the vagina.

The number of the large-diameter portions 4b provided on the columnar portion 4a is not particularly limited, and may be appropriately set and changed.

In addition, the large-diameter portions 4b do not have to be provided in a region nearer to the body portion 3 in the longitudinal direction of the columnar portion 4a, and in the range of about two thirds of the length in the longitudinal direction of the columnar portion 4a. However, since the large-diameter portions 4b are not located in the region of the distal end portion 4c of the columnar portion 4a, which is more likely to come into contact with the inner wall of the vagina when the menstrual cup 1 is held in the vagina, it is possible to reduce discomfort felt by the user.

FIGS. 3A and 3B and FIGS. 4A and 4B illustrate a plurality of rib portions provided in a region above the handle portion 4 and below the body portion 3. The rib portions are members for preventing slip when the user touches the lower portion of the body portion 3 of the menstrual cup 1 or the upper portion of the handle portion 4 with a finger. The rib portions include a first rib portion 3c formed along the entire circumference on the lower outer peripheral surface of the body portion 3 and a second rib portion 3d and a third rib portion 3e provided below the first rib portion 3c to have a circumferential length smaller than that of the first rib portion 3c (see FIGS. 3B and 4B).

Each of the portions 3c, 3d, and 3e and the third rib portion 3e protrude downwards from the outer peripheral surface of the body portion 3. In addition, transition portions 3f between the rib portions 3c, 3d, and 3e and the outer peripheral surface of the body portion 3 are formed as curved surfaces. As a result, the occurrence of tearing at each of the transition portions 3f can be suppressed, and the durability can be improved. In addition, this makes it possible to reduce discomfort felt by the user even when the respective transition portions 3f come into contact with the inner wall or the like of the vagina.

Further, since the respective rib portions 3c, 3d, and 3e are chamfered on the outer peripheral surfaces thereof, it is also possible to reduce the discomfort felt by the user even when the ribs 3c, 3d, and 3e come into contact with the inner wall or the like of the vagina.

Here, the rib portions do not have to be provided on the bottom surface of the body portion 3. However, it is preferable that the rib portions are provided on the bottom surface of the body portion 3 in view of the fact that the rib portions serve to prevent slip when the user touches the lower portion of the body portion 3 of the menstrual cup 1 and the upper portion of the handle portion 4 with a finger.

In addition, when the rib portions are provided, the number and size of the rib portions may be appropriately set and changed depending on the ease of manufacture or the feeling of use by the user.

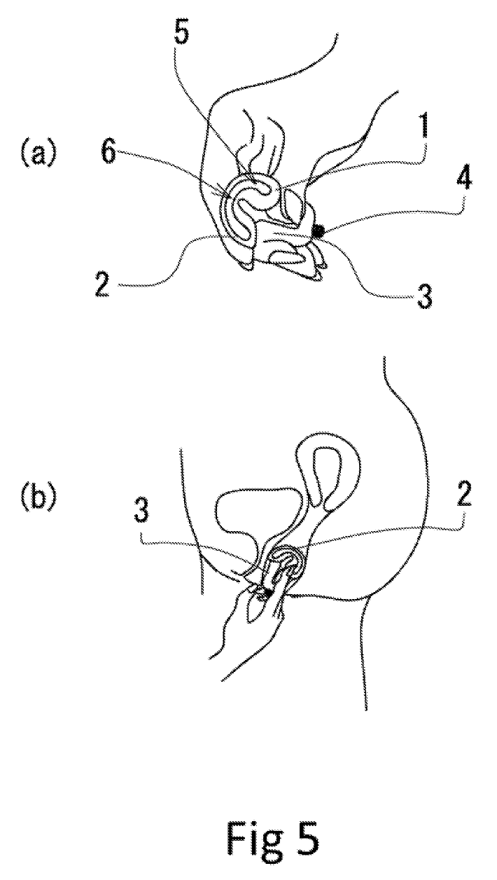

A method of using the menstrual cup 1 described above will be described with reference to FIG. 5.

(1) Insertion Method

First, the menstrual cup 1, which has been disinfected by boiling in advance, is cleansed with soap or the like, and washed by pouring enough water. At this time, the user also cleanses the fingers with soap or the like. After cleansing, the user holds the menstrual cup 1 with the handle portion 4 located downward, and crushes the outer peripheral surface of the body portion 3 from the opposite sides thereof by fingers so as to be flattened.

In addition, as illustrated in FIG. 5A, opposite ends are bent such that the shape viewed from the opening 6 side takes a U-shape (the state in which the cup is bent twice). The user sits on a toilet or takes a bent posture, and keeps the menstrual cup 1 in the bent state with a dominant hand.

The user smoothly opens the labia with the other hand and inserts the bent menstrual cup 1, from the side of the head portion 2, into the vagina from the mouth of the vagina toward the tail bone. At this time, the menstrual cup 1 is inserted while being slightly inclined in the horizontal direction.

In the state in which the menstrual cup 1 is inserted at a position slightly above the pelvic bone, the menstrual cup is located at a proper insertion position. This position is slightly lower than the insertion position of the conventional sanitary tampon. As illustrated in FIG. 5B, it is confirmed whether the menstrual cup 1 is inserted into the correct position and the body portion 3 is widened in the vagina, that is, whether the crushed shape is restored and the receiving space 5 is widened.

Confirmation as to whether the menstrual cup 1 is widened is made by holding the bottom portion of the body portion and rotating the menstrual cup 1 or by putting a finger between the vaginal wall and the menstrual cup 1 and confirming the shape of the outer peripheral surface of the body portion 3 by moving the finger along the outer peripheral surface.

(2) Method of Taking Out Menstrual Cup

Before taking out the menstrual cup 1, the user cleanses the fingers. The user finds the bottom portion of the body portion 3 while moving the thumb and index finger along the handle portion 4. When the thumb and index finger reach the bottom portion of the body portion 3, the user holds the bottom portion with the fingers and takes out air through the air holes to release the close contact with the vaginal wall. At this time, slip is prevented by the plurality of rib portions 3c, 3d, and 3e provided on the bottom portion of the body portion 3.

The user takes out the menstrual cup 1 slowly while holding the body portion 3 with fingers. The menstrual cup 1 is completely taken out and the menstrual blood collected in the receiving space 5 is discharged to the toilet or the like. After use, the user cleanses the menstrual cup 1 with soap or the like and reuses the menstrual cup 1.

The time for holding the menstrual cup 1 in the vagina should not be longer than 12 hours. The user takes out the menstrual cup 1 from the vagina 2-3 times a day, empties the menstrual cup 1, and cleanses the menstrual cup with soap or the like.

(3) Maintenance and Storage Method

After menstruation, the entire menstrual cup 1 is cleansed thoroughly with warm water. Particularly, a clogged air hole 2c is opened, thereby rendering all the air holes in the completely opened state. In addition, the entire menstrual cup 1 is thoroughly cleansed again with soap or the like, and washed thoroughly with water.

After washing, it is carefully determined whether there is no reside and the menstrual cup 1 is dried thoroughly by air. After drying, the menstrual cup 1 is stored in a case. In addition, the menstrual cup 1 is used again, the menstrual cup 1 is put in a pot or the like with enough hot water, and disinfection by boiling is performed for about 5 minutes with hot water. The disinfection may be carried out by putting the menstrual cup 1 in a dedicated container thereof and performing heating treatment in a microwave oven.

The menstrual cup 1 to which the present disclosure is applied may be used in the above-described sequence.

Since the menstrual cup to which the present disclosure is applied is capable of sufficiently receiving menstrual blood and has a structure in which the collected menstrual blood does not easily flow out to the outside, the menstrual blood does not easily leak out from the menstrual cup, and thus skin diseases are not easily caused as well. Further, since the menstrual cup is in sufficiently close contact with the vaginal wall during use and is elastically deformed, it is not easily displaced according to the movement of the user.

In addition, since the menstrual cup has a structure in which the menstrual blood collected therein does not easily come into contact with air, it is possible to prevent odor and prevent the user from experiencing sweatiness, itchiness, and the like. Also, the menstrual cup can be repeatedly used by performing disinfection and cleansing, which is also economically advantageous. In addition, the menstrual cup is capable of being easily inserted or taken out, and thus is excellent in handling property.

As described above, the menstrual cup can be used repeatedly and is of high quality with excellent feeling of use.

### DESCRIPTION OF REFERENCE NUMERALS

1 Menstrual cup
2 Head portion
2a Taper
2c Air hole
3 Body portion
3a Inner peripheral surface of the body portion
3b Reference indication portion
3c First rib portion
3d Second rib portion
3e Third rib portion
3f Transition portion between the rib portions 3c, 3d, and 3e and the outer peripheral surface of the body portion
4 Handle portion
4a Columnar portion
4b Large-diameter portion
4c Distal end portion of the columnar portion
4d Transition portion between the columnar portion and the large-diameter portion
4e Transition portion between the columnar portion and the bottom surface of the body portion
5 Receiving space
6 Opening
7 Inner wing portion
7a Cutout portion
7b Distal end portion of the inner wing portion
7c Return portion

1. A menstrual cup having a bell-shaped main body having a bottom portion, the menstrual cup comprising:
a body portion including an opening formed therein wherein the body portion extends from the bottom portion of the main body;
a head portion constructed to extend from an end of the body portion wherein an outer diameter of the head portion extends away from the body portion;
an inner wing portion constructed to cover an inner wall of a distal end portion of the head portion and constructed to protrude substantially towards the bottom portion, wherein a cutout portion is formed in at least a portion of a distal end portion of the inner wing portion wherein the distal end portion of the inner wing portion is approximate to the distal end portion of the head portion; and
a handle portion constructed to extend from the bottom portion and protrude in a direction opposite to the body portion.

2. The menstrual cup of claim 1, wherein at least one through-hole is formed in at least one of the body portion and the head portion wherein the through-hole penetrates from an outer peripheral surface to an inner peripheral surface.

3. The menstrual cup of claim 1, wherein a plurality of convex portions are formed on an outer peripheral surface of the handle portion in a longitudinal direction with respect to the handle portion.

4. The menstrual cup of claim 3, wherein the plurality of convex portions are formed in a region of the hand portion at a side of the body portion and corresponding to two thirds of a length of the handle portion in the longitudinal direction with respect to the handle portion.

5. The menstrual cup of claim 1, wherein a ratio of the length of the handle portion in the longitudinal direction and a length of the body portion and the head portion in the longitudinal direction is in a range of 1:3.0 to 1:4.5.

6. The menstrual cup of claim 1, wherein a rib portion is provided on an outer peripheral surface about the bottomed bore portion, and the rib portion protrudes outwardly.

7. (canceled)
